SEQUENCE LISTING (51 sequences)

SEQ ID NO: 1 - DNA, 26 bp, Artificial Sequence
  Primer
tttttcatattttattgtaacaaacm

SEQ ID NO: 2 - DNA, 36 bp, Artificial Sequence
  Primer
cctagtgtaagtggcacccaatccgtccacctgtgr

SEQ ID NO: 3 - DNA, 31 bp, Artificial Sequence
  Primer
ctgctcctgtaaagtgacctgttcctgaagy

SEQ ID NO: 4 - DNA, 26 bp, Artificial Sequence
  Primer
tacagtgttacaattaaaaactgatr

SEQ ID NO: 5 - DNA, 26 bp, Artificial Sequence
  Primer
ccaaaccagattagtatgtagttagy

SEQ ID NO: 6 - DNA, 41 bp, Artificial Sequence
  Primer
aataagaacctcagagctctttaaataatttctcacaatgy

SEQ ID NO: 7 - DNA, 41 bp, Artificial Sequence
  Primer
ctgaccggtttagtactactcccgtatcactattagtaack

SEQ ID NO: 8 - DNA, 36 bp, Artificial Sequence
  Primer
ttatattaagtgtaaaataagatgtcttttattcay

SEQ ID NO: 9 - DNA, 21 bp, Artificial Sequence
  Primer
catgcaattcaggaccgtgca

SEQ ID NO: 10 - DNA, 24 bp, Artificial Sequence
  Primer
gtggacacccatttgtaccctatg

SEQ ID NO: 11 - DNA, 31 bp, Artificial Sequence
  Primer
gttccagagaaatggaattatatttgttttm

SEQ ID NO: 12 - DNA, 21 bp, Artificial Sequence
  Primer
gatgtatttgtgtagaactcr

SEQ ID NO: 13 - DNA, 26 bp, Artificial Sequence
  Primer
gctaaagatagaaaaagcaaattctr

SEQ ID NO: 14 - DNA, 36 bp, Artificial Sequence
  Primer
aagtcagtcactataataaaggcaaatcttcccagr

SEQ ID NO: 15 - DNA, 28 bp, Artificial Sequence
  Primer
tctgtaggaaaggaatctgtatggaaar

SEQ ID NO: 16 - DNA, 34 bp, Artificial Sequence
  Primer
gctctgtgcttaaaaggatactctgatttggaay

SEQ ID NO: 17 - DNA, 25 bp, Artificial Sequence
  Primer
ttaagctctctgagtgtacttttaw

SEQ ID NO: 18 - DNA, 32 bp, Artificial Sequence
  Primer
caggacgtatcgacaggaaaatagagttccck

SEQ ID NO: 19 - DNA, 36 bp, Artificial Sequence
  Primer
acctggtcatgataaacttcatgcaacttcactacr

SEQ ID NO: 20 - DNA, 31 bp, Artificial Sequence
  Primer
gggtccccctcccagaggctttgggtggccy

SEQ ID NO: 21 - DNA, 26 bp, Artificial Sequence
  Primer
gagagaagaaaatgttcttattaggy

SEQ ID NO: 22 - DNA, 21 bp, Artificial Sequence
  Primer
gttcatcatcaaacagtgcar

SEQ ID NO: 23 - DNA, 46 bp, Artificial Sequence
  Primer
atgtggatccagatgatgacgaggtggtgggctgaatagtggaggy

SEQ ID NO: 24 - DNA, 41 bp, Artificial Sequence
  Primer
tagacagcagtaagtgacaaagtatctgcctaaatcagctr

SEQ ID NO: 25 - DNA, 41 bp, Artificial Sequence
  Primer
caggaaagaagcttcctcctggtggaatacctggcattgay

SEQ ID NO: 26 - DNA, 44 bp, Artificial Sequence
  Primer
gagaacaaaatgtttcaaatgttgtcagagtagacctggagtar

SEQ ID NO: 27 - DNA, 21 bp, Artificial Sequence
  Primer
gttgctgagagcctggtgcag

SEQ ID NO: 28 - DNA, 22 bp, Artificial Sequence
  Primer
cctgtatgtggaattacttctc

SEQ ID NO: 29 - DNA, 36 bp, Artificial Sequence
  Primer
catgctctagcccttaatattttcaaatgttagtcy

SEQ ID NO: 30 - DNA, 21 bp, Artificial Sequence
  Primer
atggctgttcaaaagtaaccr

SEQ ID NO: 31 - DNA, 21 bp, Artificial Sequence
  Primer
gcatcctccaaagccattgcy

SEQ ID NO: 32 - DNA, 26 bp, Artificial Sequence
  Primer
attctgtgattctatgattctaacar

SEQ ID NO: 33 - DNA, 49 bp, Artificial Sequence
  Primer
tcagataaactcttgcatagtttctcagttgatttagctccttatctcr

SEQ ID NO: 34 - DNA, 36 bp, Artificial Sequence
  Primer
agccgaagcctcttgaggacttctccaaacttctcr

SEQ ID NO: 35 - DNA, 41 bp, Artificial Sequence
  Primer
tgattaagtgctactaagtatcatacaccttatgatttgcy

SEQ ID NO: 36 - DNA, 31 bp, Artificial Sequence
  Primer
ttctacttctttcttgcaaaaataaactcaw

SEQ ID NO: 37 - DNA, 36 bp, Artificial Sequence
  Primer
agtttgtgagcatactgttactctttagatttcaty

SEQ ID NO: 38 - DNA, 26 bp, Artificial Sequence
  Primer
aatatgtacatcatgagagcttgacr

SEQ ID NO: 39 - DNA, 21 bp, Artificial Sequence
  Primer
atctcagccccataaaaacgy

SEQ ID NO: 40 - DNA, 20 bp, Artificial Sequence
  Primer
tgccagcccgtaatcatagg

SEQ ID NO: 41 - DNA, 20 bp, Artificial Sequence
  Primer
aagcaccacgacccaatcta

SEQ ID NO: 42 - DNA, 21 bp, Artificial Sequence
  Primer
ctgctttctgctctcgagttr

SEQ ID NO: 43 - DNA, 26 bp, Artificial Sequence
  Primer
tgtatgccaacaccaaccgataccay

SEQ ID NO: 44 - DNA, 21 bp, Artificial Sequence
  Primer
atctcagccccataaaaacgc

SEQ ID NO: 45 - DNA, 21 bp, Artificial Sequence
  Primer
tagagtcggggtatttttgcg

SEQ ID NO: 46 - DNA, 21 bp, Artificial Sequence
  Primer
atctcagccccataaaaacgt

SEQ ID NO: 47 - DNA, 21 bp, Artificial Sequence
  Primer
tagagtcggggtatttttgca

SEQ ID NO: 48 - DNA, 20 bp, Artificial Sequence
  Primer
acggtcgggcattagtatcc

SEQ ID NO: 49 - DNA, 20 bp, Artificial Sequence
  Primer
ttcgtggtgctgggttagat

SEQ ID NO: 50 - DNA, 19 bp, Artificial Sequence
  Primer
gcattgctcctcattcaga

SEQ ID NO: 51 - DNA, 21 bp, Artificial Sequence
  Primer
tgtaaaagagcagggtcattg

(19) United States
(12) Patent Application Publication
(10) Pub. No.: US 2012/0102583 A1
(43) Pub. Date: Apr. 26, 2012

(54) RESISTANCE TO BACTERIAL INFECTION

(75) Inventors: Mark Fife, Pewsey (GB); Peter Kaiser, Lauder (GB); Nigel Salmon, Wantage (GB)

(73) Assignee: INSTITUTE FOR ANIMAL HEALTH, PIRBRIGHT (GB)

(21) Appl. No.: 13/266,366

(22) PCT Filed: Apr. 28, 2010
(86) PCT No.: PCT/GB10/00850

§ 371 Date: Jan. 17, 2012

(30) Foreign Application Priority Data
Apr. 29, 2009 (GB) 0907409.7

(51) Int. Cl. C12Q 1/68; C07H 21/04; A01K 67/027

(52) U.S. Cl. 800/21; 435/6.11; 536/24.33; 536/24.31

(57) ABSTRACT

The present invention provides a method of identifying an animal having a genotype associated with resistance to bacterial infection comprising the steps of: (a) providing a sample from said mammal; (b) determining the alleles at one or more markers of the SAL1 locus to identify the genotype of the marker, wherein said SAL1 locus lies between 54.0 MB to 54.8 MB of chicken Chromosome 5 or an equivalent thereof; and (c) determining whether the genotype is a genotype associated with resistance to bacterial infection.

### FIELD OF INVENTION

The present invention relates to methods of identifying animals having a genotype associated with resistance to bacterial infection and, optionally, selecting those animals having a genotype associated with resistance to bacterial infection. Further, the present invention relates to methods for predicting the response of animals to infection by bacteria. In addition, the present invention relates to methods for producing animals which are resistant to bacterial infection or increasing the resistance to bacterial infection and the present invention relates to animals produced by said method.

The present invention also relates to the use of one or more markers at the SAL1 locus for identifying and, optionally, selecting animals with a genotype associated with resistance to bacterial infection. Additionally, the present invention relates to the use of one or more markers at the SAL1 locus for predicting the response of an animal to infection with bacteria.

The present further relates to kits for identifying in a sample the genotype of one or more markers at the SAL1 locus; arrays; and isolated oligonucleotide primers or probes.

### BACKGROUND TO THE INVENTION

The bacterial infection of animals, such as domestic fowl, is a common problem in animal husbandry and can result in substantial losses of livestock. Moreover, the presence and control of bacterial infections in animals, in order to reduce the food-borne infections of humans, is an important public health issue. Examples of bacterial infections which can have a significant economic impact on animal husbandry and which, if not controlled, can cause food-poisoning in humans include: infection by Salmonella, Campylobacter (such as Campylobacter jejuni and Campylobacter coli), Clostridium (such as Clostridium perfringens) and Staphylococcus (such as Staphylococcus aureus).

For example, around 10,000-30,000 cases of human salmonellosis were reported per annum in England and Wales alone in the last 10 years (Health Protection Agency, 2008—www.hpa.org.uk). The consumption of infected poultry meat and eggs is a major source of human cases. Therefore, the presence and control of Salmonella infections in chicken flocks remains an important public health issue. In addition to the potential for human disease some extremely pathogenic serotypes that are highly adapted to animal hosts, such as S. enterica serovar Gallinarum in poultry, can cause severe disease often resulting in whole flock death. The increased resistance to antibiotics is an inevitable side effect of their continued use, which has resulted in the European Community forcing poultry producers to minimize salmonella contamination in breeders and layers (Zoonosis Directive EC/92/117). In addition, the prophylactic use of antimicrobials in poultry production was banned in 2006 by the European Commission, except under very limited circumstances, such as on animal health and welfare grounds in order to minimise the development of antibiotic resistance.

Extensive analysis of inbred chicken lines revealed that some lines are consistently either susceptible or resistant to many serovars (i.e. types) of Salmonella, indicating a common resistance mechanism (Bumstead and Barrow 1993; Kaiser and Lamont 2001). Furthermore, the resistance in lines W1, 6₁ and N were also shown to be dominant (consistent with inheritance of a major quantitative trait locus—QTL) and neither sex—nor MHC-linked (Wigley, Hulme et al. 2002). Resistant birds show resistance to both oral and intramuscular infection, however, the difference is most pronounced in intravenous infection of young chicks, with susceptible birds succumbing to a dose of less than 10 cfu of Salmonella typhimurium (Bumstead and Barrow 1993).

Studies in mice have established extensive genetic differences in salmonellosis susceptibility and have identified numerous candidate genes involved in disease resistance, including Slc11a1 (formerly Nramp1), NOS and TLR-4 (Poltorak, Smimova et al. 1998; Ables, Takamatsu et al. 2001; O'Brien, Wang et al. 2005). Although Slc11a1, TRAIL, TGFb2 & TGFb3, PSAP, TLR4 (Hu, Bumstead et al. 1997; Sebastiani, Olien et al. 1998; Leveque, Forgetta et al. 2003; Malek and Lamont 2003) and the MHC complex (Zhou and Lamont 2003) have been implicated in resistance to salmonella colonisation in the chicken, they show no significant association with resistance to salmonellosis in the line 6₁×15I cross (Mariani, Barrow et al. 2001). It is therefore evident that other, as yet unidentified genes are involved in this multifactorial trait.

Mariani, Barrow et al. (2001) identified significant linkage to a region of chicken Chromosome 5 (Chr 5), designated SAL1, for salmonellosis disease resistance. In this investigation, a first generation backcross of line 15I and line 6₁ was used to map disease resistance for salmonellosis, which showed a considerable effect that was consistently observed over three separate experiments. This region on chicken Chr 5 shows conserved synteny with Human Chr 14 and mouse Chr 12. Within this region lie two genes which were identified as potential candidates, creatine kinase (CKB) and dynein (DNCH1) (Mariani, Barrow et al. 2001). However, due to the limited number of meioses in this first generation backcross, and the use of only widely spaced microsatellite markers, the mapping resolution was insufficient to warrant further characterisation of these prospective candidates (Mariani, Barrow et al. 2001). Additional evidence for a salmonellosis resistance locus within this region of Chr 5 has since been shown. In a combined F₂ and Backcross study examining Salmonella enteritidis colonization in chickens, Tilquin et al (2005) identified seven highly significant QTLs. One of these was SAL1, located at about 150 cM on Chr 5, and was confirmed in both data sets with p=0.0514 and 0.0034 in the F₂ and backcross, respectively (Tilquin, Barrow et al. 2005). In an independent study Kaiser and Lamont (2002) observed an association of the microsatellite ADL0298 (˜198 cM, on Chr 5) with Salmonella enteritidis levels in the caeca and spleen one week after oral inoculation of day-old chicks (Kaiser and Lamont 2002). This significant QTL, albeit ˜48 cM from the previously identified SAL1 locus, was identified using only an F1 cross and a very limited set of microsatellite markers. The limited power to resolve a QTL using this design could explain the poor resolution of the QTL.

The chicken genome comprises over a billion base pairs of which at least 3 million positions are polymorphic (Wong, Liu et al. 2004). These sequence variations can result in phenotypic differences, such as differential resistance to disease, or are used as markers because of close proximity to the causative gene. The challenge is to locate the gene of interest and determine the nature of the allele(s) that contributes to disease resistance.

### SUMMARY OF INVENTION

The present inventors have found that by using a sixth generation backcross population and a mapping approach combining densely packed SNP and microsatellite markers they were able to refine the SAL1 locus of chicken Chromosome 5. The present inventors show that the SAL1 locus lies between 54.0-54.8 MB on the long arm of chicken Chromosome 5. Furthermore, the present inventors have identified potential positional candidate genes which lie within the refined SAL1 locus.

In one aspect, the present invention provides a method of identifying an animal having a genotype associated with resistance to bacterial infection or a genotype associated with susceptibility to bacterial infection comprising the steps of:
(a) providing a sample from said animal;
(b) determining the alleles at one or more markers of the SAL1 locus to identify the genotype of the marker, wherein said SAL1 locus lies between 54.0 MB to 54.8 MB of chicken Chromosome 5 or an equivalent thereof; and
(c) determining whether the genotype is (i) a genotype associated with resistance to bacterial infection or (ii) a genotype associated with susceptibility to bacterial infection.

In a further aspect, the present invention provides a method of identifying a genotype associated with resistance to bacterial infection or a genotype associated with susceptibility to bacterial infection comprising the steps of
(a) providing samples from more than one animal;
(b) determining that there is an allelic variant at a marker of the SAL1 locus to identify a polymorphic marker, wherein said SAL1 locus lies between 54.0 MB to 54.8 MB of chicken Chromosome 5 or an equivalent thereof;
(c) determining that a genotype of the polymorphic marker is associated with resistance to bacterial infection; or
(d) determining that a genotype of the polymorphic marker is associated with susceptibility to bacterial infection.

There is provided, in another aspect of the present invention, a method for predicting the response of an animal to infection by bacteria comprising the steps of:
(a) providing a sample from said animal;
(b) determining the alleles at one or more markers of the SAL1 locus to identify the genotype of the marker, wherein said SAL1 locus lies between 54.0 MB to 54.8 MB of chicken Chromosome 5 or an equivalent thereof; and
(c) determining whether the genotype is: (i) a genotype associated with resistance to bacterial infection, or (ii) a genotype associated with susceptibility to bacterial infection, to predict the response of an animal to infection by bacteria.

The present invention provides, in another aspect, a method for producing an animal which is resistant to bacterial infection or increasing the resistance to bacterial infection of an animal wherein said method comprises the step of replacing at least part of the SAL1 locus with a SAL1 locus or corresponding part thereof from an animal which is resistant to bacterial infection, wherein the SAL1 locus lies between 54.0 MB to 54.8 MB of chicken Chromosome 5 or an equivalent thereof.

In another aspect, the present invention provides an animal which is resistant to bacterial infection by replacing at least part of the SAL1 locus with a SAL1 locus or corresponding part thereof from an animal which is resistant to bacterial infection, wherein the SAL1 locus lies between 54.0 MB to 54.8 MB of chicken Chromosome 5 or an equivalent thereof.

In a further aspect, the present invention provides a method for producing an animal which is susceptible to bacterial infection or increasing the susceptibility to bacterial infection of an animal wherein said method comprises the step of replacing at least part of the SAL1 locus with a SAL1 locus or corresponding part thereof from an animal which is susceptible to bacterial infection, wherein the SAL1 locus lies between 54.0 MB to 54.8 MB of chicken Chromosome 5 or an equivalent thereof.

In another aspect, the present invention provides an animal which is susceptible to bacterial infection by replacing at least part of the SAL1 locus with a SAL1 locus or corresponding part thereof from an animal which is susceptible to bacterial infection, wherein the SAL1 locus lies between 54.0 MB to 54.8 MB of chicken Chromosome 5 or an equivalent thereof.

Further, the present invention provides the use of one or more markers at the SAL1 locus for identifying (i) an animal with a genotype associated with resistance to bacterial infection or (ii) an animal with a genotype associated with susceptibility to bacterial infection; wherein said SAL1 locus lies between 54.0 MB to 54.8 MB of chicken Chromosome 5 or an equivalent thereof.

The present invention further provides the use of one or more markers at the SAL1 locus for selecting (i) an animal with a genotype associated with resistance to bacterial infection or (ii) an animal with a genotype associated with susceptibility to bacterial infection; wherein said SAL1 locus lies between 54.0 MB to 54.8 MB of chicken Chromosome 5 or an equivalent thereof.

In another aspect, the present invention provides the use of one or more markers at the SAL1 locus for predicting the response of an animal to infection with bacteria.

In another aspect, the present invention provides a kit for identifying in a sample the genotype of one or more markers at the SAL1 locus, wherein said SAL1 locus lies between 54.0 MB to 54.8 MB of chicken Chromosome 5 or an equivalent thereof, wherein said kit comprises a means for determining alleles of one or more markers.

In a further aspect, the present invention provides a kit for identifying in a sample the genotype of one or more markers at the SAL1 locus, wherein said SAL1 locus lies between 54.0 MB to 54.8 MB of chicken Chromosome 5 or an equivalent thereof, and wherein said kit comprises a means for determining alleles of one or more markers wherein said one or more markers are selected from the group consisting of:
the single nucleotide polymorphism SNP2;
the microsatellite marker ADL166;
a polymorphism in the nucleotide sequence ENSGALG00000011620 encoding AKT(1); and
a polymorphism in the nucleotide sequence ENSGALG00000011619 encoding CD-27 binding protein.

In another aspect, the present invention provides for the use of a kit mentioned herein.

The present invention provides, in a further aspect, an array wherein said array comprises one or more oligonucleotide probes capable of determining in a sample the alleles at one or more markers at the SAL1 locus, wherein said SAL1 locus lies between 54.0 MB to 54.8 MB of chicken Chromosome 5 or an equivalent thereof.

The present invention provides, in another aspect, an array wherein said array comprises one or more oligonucleotide probes capable of determining in a sample the alleles at one or more markers wherein said one or more markers are selected from the group consisting of:
the single nucleotide polymorphism SNP2;
the microsatellite marker ADL166;
a polymorphism in the nucleotide sequence ENSGALG00000011620 encoding AKT(1); and
a polymorphism in the nucleotide sequence ENSGALG00000011619 encoding CD-27 binding protein.

The present invention provides, in a further aspect, an isolated oligonucleotide primer or oligonucleotide probe wherein said oligonucleotide probe or oligonucleotide primer is selected from the group consisting of SEQ ID Nos 39 to 41 and 44 to 49.

### DESCRIPTION OF FIGURES

FIG. 1. SNP and microsatellite markers used in the mapping analysis of the SAL1 locus to refine the SAL1 locus. SNP interval units are shown as recombination distances in centiMorgans (cM) calculated from the average recombination rate across the published genomic sequence for chicken chromosome 5.

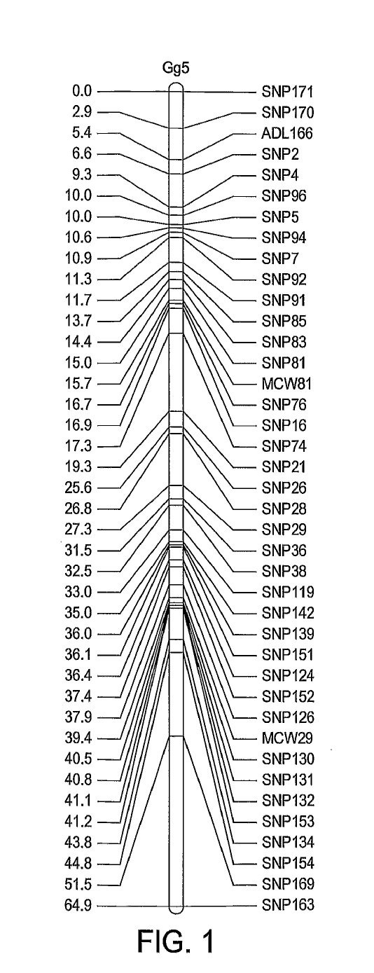

FIG. 2. Interval mapping analysis of log, transformed bacterial counts for 40 markers flanking the SAL1 locus on chicken chromosome 5. The predicted QTL location 4.8-6.2 cM has a highly significant association (P=0.0047). Significance levels were calculated by permutation analysis using 1000 permutations at 1 cM intervals. The lower horizontal line is the P<0.05 and the upper line P<0.01 level of significance. Candidate genes with genomic positions based on Gallus gallus (chicken) Build 2.1 within the refined SAL1 locus (54.0-54.8 MB) are indicated.

### DETAILED DESCRIPTION

The method of identifying an animal having a genotype associated with resistance to bacterial infection as mentioned herein may further comprise the step of:
(d) selecting an animal having the genotype associated with resistance to bacterial infection.

As mentioned herein, the method of identifying an animal having a genotype associated with susceptibility to bacterial infection may further comprise the step of:
(d) selecting an animal having the genotype associated with susceptibility to bacterial infection.

### Bacterial Infection

As used herein the phrases “resistant to bacterial infection” and “resistance to bacterial infection” refer to an animal in whom: the frequency of infection by a type of bacteria in a given time period is lower than the average frequency of infection (i.e. mean number of infections) in the general population in a given time period (such as in a three-month period or in a six-month period); and/or the severity of infection by a type of bacteria over a given time period is lower than the average severity of infection (i.e. the mean severity of infection) in the general population over a given time period (such as over day 1 post-infection, or days 1 and 2 post-infection, or days 1 to 3 post-infection or days 1 to 5 post-infection); and/or the length of time it takes for a bacterial infection to clear (in the absence of treatment with antimicrobials) is shorter than the average time taken in the general population; and/or the extent of the bacterial infection (such as the bacterial count in blood serum and/or the number of organs infected and/or the severity of infection, measured by Quantitative PCR to detect levels of bacterial proliferation) at a given time point after infection (such as 2 days post-infection) is less than the average in the general population. In order to carry out such comparisons, the animals must be subject to the same environmental conditions in order to minimise factors other than genotype effecting the progression of infection.

The bacterial infection may be an infection by one or more bacteria which are capable of causing food-poisoning in humans—in other words, the one or more bacteria are capable of causing a food-borne disease. The bacterial infection may be, for example, an infection by one or more bacteria selected from the group consisting of Salmonella, Campylobacter, Clostridium and Staphylococcus. In one example, the bacterial infection is an infection by Salmonella and/or Campylobacter. In another example, the bacterial infection is an infection by Salmonella.

Bacterial infections by Salmonella and Campylobacter account for a significant number of food poisoning cases associated with chicken.

The bacterial infection may be an infection by one or more strains of a bacterium.

Examples of Salmonella strains capable of causing food-poisoning in humans include: Salmonella enteritidis (such as Salmonella enterica subsp. enterica serovar Typhimurium and Salmonella enterica subsp. enterica serovar Enteritidis, Salmonella enterica serotype Typhi), Salmonella serovar Saintpaul, and Salmonella Rissen.

Infection of an animal by Salmonella may cause salmonellosis in said animal. The term “salmonellosis” as used herein refers to infection with or disease caused by bacteria of the genus Salmonella. Salmonellosis is typically marked by gastroenteritis but may be complicated by septicaemia, meningitis, endocarditis, and various focal lesions (such as in the kidneys). In humans, salmonellosis is characterized by the sudden onset of abdominal pain, vomiting, diarrhoea, and fever.

In one example, a genotype associated with resistance to bacterial infection is a genotype associated with resistance to salmonellosis or Salmonella infection.

Examples of Campylobacter strains capable of causing food-poisoning in humans include Campylobacter jejuni and Campylobacter coli.

In humans, Campylobacter may cause gastroenteritis, causing diarrhoea, stomach cramps and in rare cases a nervous condition called Guillain-Barré syndrome.

In another example, a genotype associated with resistance to bacterial infection is a genotype associated with resistance to Campylobacter infection such as Campylobacter jejuni and/or Campylobacter coli.

Examples of Clostridium strains capable of causing food-poisoning in humans include Clostridium perfringens.

In humans Clostridium may cause diarrhoea and severe abdominal pain.

In a further example, a genotype associated with resistance to bacterial infection is a genotype associated with resistance to Clostridium infection such as Clostridium perfringens infection.

Examples of Staphylococcus strains capable of causing food-poisoning in humans include Staphylococcus aureus.

In humans, Staphylococcus may cause gastroenteritis causing nausea, vomiting, stomach cramps, and diarrhoea.

In a further example, a genotype associated with resistance to bacterial infection is a genotype associated with resistance to Staphylococcus infection such as Staphylococcus aureus infection.

As used herein the phrases “susceptibility to bacterial infection” and “susceptible to bacterial infection” refer to an animal in whom: the frequency of infection by a type of bacteria in a given time period is higher than the average frequency of infection (i.e. mean number of infections) in the general population in a given time period (such as in a three-month period or in a six-month period); and/or the severity of infection by a type of bacteria over a given time period is higher than the average severity of infection (i.e. the mean severity of infection) in the general population over a given time period (such as over day 1 post-infection, or days 1 and 2 post-infection, or days 1 to 3 post-infection or days 1 to 5 post-infection); and/or the length of time it takes for a bacterial infection to clear (in the absence of treatment with antimicrobials) is longer than the average time taken in the general population; and/or the extent of the bacterial infection (such as the bacterial count in blood serum and/or the number of organs infected and/or the severity of infection, measured by Quantitative PCR to detect levels of bacterial proliferation) at a given time point after infection (such as 2 days post-infection) is greater than the average in the general population. In order to carry out such comparisons, the animals must be subject to the same environmental conditions in order to minimise factors other than genotype effecting the progression of infection. Individuals “susceptible to bacterial infection” are not, however, immune-compromised individuals as they do not show an increased susceptibility to, for example, viral infections when compared to the general population.

### Genotypes

The term “genotype” as used herein refers to the set of alleles present in an individual at one or more markers mentioned herein. At any one autosomal locus, a genotype will be either homozygous (with two identical alleles) or heterozygous (with two different alleles).

As used herein, the term “allele” refers to a given form (i.e. type) of a marker on a chromosome. In a diploid cell or organism, the two alleles of a given marker typically occupy corresponding loci on a pair of homologous chromosomes.

The alleles, and thus the genotype, of an individual for a specific marker can be determined using recombinant DNA techniques such as PCR, DNA sequencing, hybridization, ASO probes, and hybridization to DNA microarrays or beads.

The samples used in order to determine the alleles at a marker (i.e. to genotype the animal) comprise genomic DNA.

The term “polymorphism” as used herein refers to the occurrence of two or more distinct forms (types) of alleles at a marker—in other words, variants.

A polymorphism at a marker may be identified by using recombinant DNA techniques such as PCR, DNA sequencing and hybridization.

### Markers of the SAL1 Locus

The term “marker” used in the phrase “one or more markers of the SAL1 locus” herein refers to a feature of the genome (e.g., a nucleotide or a polynucleotide sequence that is present in the genome) that lies in the SAL1 locus. The markers used in the methods described herein are polymorphic markers—e.g. the markers have at least two distinct types of alleles.

Examples of types of markers include, single nucleotide polymorphisms (SNPs), indels (i.e., insertions/deletions), simple sequence repeats (SSRs), restriction fragment length polymorphisms (RFLPs), random amplified polymorphic DNAs (RAPDs), cleaved amplified polymorphic sequence (CAPS) markers, Diversity Arrays Technology (DArT) markers, and amplified fragment length polymorphisms (AFLPs), Microsatellites or Simple sequence repeat (SSRs) among many other examples. Markers can, for example, be used to locate genetic loci containing alleles that contribute to variability in expression of phenotypic traits on a chromosome.

One or more markers which reside in the SAL1 locus may be used in the methods described herein. For example, two or more markers in the SAL1 locus may be used in the methods described herein. Further, three or more markers in the SAL1 locus may be used in the methods described herein.

The term “SAL1 locus” as used herein refers to a quantitative trait locus (QTL). The SAL1 locus is a region of the genome which is associated (i.e. linked) with having an effect on the progression of bacterial infections, such as Salmonella, in an animal. In some animals, the SAL1 locus is associated with resistance to bacterial infection. In other animals the SAL1 locus is associated with susceptibility to bacterial infection.

In chickens (Gallus gallus), for instance, the SAL1 locus lies on the long arm of Chromosome 5 between 54.0 to 54.8 MB on the long arm of Chromosome 5.

In other animals, the SAL1 locus lies in a region equivalent to the SAL1 locus on chicken Chromosome 5. For example, human Chromosome 14 and mouse Chromosome 12 show conserved synteny with 54.0 to 54.8 MB of chicken Chromosome 5; thus the equivalent of the chicken SAL1 locus in humans lies on human Chromosome 14 and the equivalent of the chicken SAL1 locus in mice lies on Chromosome 12.

Thus the term “or an equivalent thereof” in the phrase “wherein said SAL1 locus lies between 54.0 MB to 54.8 MB of chicken Chromosome 5 or an equivalent thereof” refers to the chromosomal region of an animal other than a chicken which has conserved synteny with 54.0 MB to 54.8 MB of chicken Chromosome 5. Typically, the order of genes in the chromosome region of said equivalent is similar or the same as in 54.0 MB to 54.8 MB of chicken Chromosome 5.

Examples of markers at the SAL1 locus on chicken Chromosome 5 include but are not limited to:
the single nucleotide polymorphism SNP2 (rs16511470);
the microsatellite marker ADL166 (UniSTS:71823);
a polymorphism in the nucleotide sequence ENSGALG00000011620 (AKT1);
a polymorphism in the nucleotide sequence ENSGALG00000011619 (SIVA1);
a polymorphism in the nucleotide sequence ENSGALG00000011698;
a polymorphism in the nucleotide sequence ENSGALG00000011696;
a polymorphism in the nucleotide sequence ENSGALG00000020365;
a polymorphism in the nucleotide sequence ENSGALG00000011692;
a polymorphism in the nucleotide sequence ENSGALG00000023023;
a polymorphism in the nucleotide sequence ENSGALG00000011690;
a polymorphism in the nucleotide sequence ENSGALG00000011687;
a polymorphism in the nucleotide sequence ENSGALG00000011656;
a polymorphism in the nucleotide sequence ENSGALG00000011646;
a polymorphism in the nucleotide sequence ENSGALG00000011639;
a polymorphism in the nucleotide sequence ENSGALG00000023025;
a polymorphism in the nucleotide sequence ENSGALG00000011618; and
a polymorphism in the nucleotide sequence ENSGALG00000011608.

Some markers mentioned herein may also be referred to herein as “candidate genes”. The term “candidate gene” as used herein refers to any marker which lies within the SAL1 locus (54.0 MB to 54.8 MB of chicken Chromosome 5 or an equivalent thereof) which may encode a polypeptide sequence. The candidate gene may have a role in causing resistance/susceptibility to bacterial infection (such as Salmonella infection).

In one example, the markers at the SAL1 locus on chicken Chromosome 5 are:
the single nucleotide polymorphism SNP2 (rs16511470);
the microsatellite marker ADL166 (UniSTS:71823);
a polymorphism in the nucleotide sequence ENSGALG00000011620 (AKT1); and
a polymorphism in the nucleotide sequence ENSGALG00000011619 (SIVA1).

In another example, the marker at the SAL1 locus on chicken Chromosome 5 is:
the single nucleotide polymorphism SNP2 (rs16511470); or
the microsatellite marker ADL166 (UniSTS:71823).

In a further example, the markers at the SAL1 locus on chicken Chromosome 5 are:
the single nucleotide polymorphism SNP2 (rs16511470); and
the microsatellite marker ADL166 (UniSTS:71823).

The single nucleotide polymorphism SNP2 on chicken Chromosome 5 mentioned herein has either the nucleotide C or the nucleotide T. Said SNP has the universal identifier rs16511470.

The microsatellite marker ADL166 on chicken Chromosome 5 is a di-nucleotide (TG) x15 repeat (PCR product size: 135-156 (bp), Gallus gallus). Said microsatellite has the universal identifier UniSTS:71823.

The term “nucleotide sequence ENSGALG00000011620” as used herein refers to a polynucleotide sequence at nucleotides 54122670 to 54193661 on chicken Chromosome 5. The polynucleotide sequence may also be referred to as AKT1. The polynucleotide sequence encodes the polypeptide AKT(1). The polypeptide sequence encoded by the polynucleotide sequence may also be referred to as v-akt murine thymoma viral oncogene homolog 1.

One example of a polymorphism in the nucleotide sequence ENSGALG00000011620 (AKT1) is the microsatellite marker ADR006 (forward primer: GCATTGCTCCTCATTCAGA—SEQ ID NO 50—and reverse primer: TGTAAAAGAGCAGGGTCATTG—SEQ ID NO 51; PCR product size: about 196 bp Gallus gallus). The microsatellite marker ADR006 on chicken Chromosome 5 has the universal identifier UniSTS:462634.

The term “nucleotide sequence ENSGALG00000011619” as used herein refers to a polynucleotide sequence at nucleotides 54107622 to 54109526 on chicken Chromosome 5. The polynucleotide sequence may also be referred to as SIVA1. The polynucleotide sequence encodes CD27-binding (Siva) protein.

The term “nucleotide sequence ENSGALG00000011698” as used herein refers to a polynucleotide sequence at nucleotides 54739263 to 54790063 on chicken Chromosome 5. The polynucleotide sequence may also be referred to as NUDT14. The polynucleotide sequence encodes a polypeptide similar to UDPG pyrophosphatase (EC 3.6.1.45). The polypeptide sequence encoded by the polynucleotide sequence may also be referred to as nudix (nucleoside diphosphate linked moiety X)-type motif 14.

The term “nucleotide sequence ENSGALG00000011696” as used herein refers to a polynucleotide sequence at nucleotides 54641798 to 54703903 on chicken Chromosome 5. The polynucleotide sequence encodes a polypeptide similar to C-Serrate-2.

The term “nucleotide sequence ENSGALG00000020365” as used herein refers to a polynucleotide sequence at nucleotides 54495759 to 54496625 on chicken Chromosome 5. The polynucleotide sequence encodes the polypeptide ‘Probable G-protein coupled receptor 132’.

The term “nucleotide sequence ENSGALG00000011692” as used herein refers to a polynucleotide sequence at nucleotides 54472833 to 54473582 on chicken Chromosome 5. The polynucleotide sequence encodes the polypeptide ‘cell division cycle associated 4’.

The term “nucleotide sequence ENSGALG00000023023” as used herein refers to a polynucleotide sequence at nucleotides 54456824 to 54457755 on chicken Chromosome 5. The polynucleotide sequence encodes a polypeptide similar to Transcriptional regulator TRIP-Br2.

The term “nucleotide sequence ENSGALG00000011690” as used herein refers to a polynucleotide sequence at nucleotides 54442595 to 54450587 on chicken Chromosome 5. The polynucleotide sequence encodes a polypeptide similar to the BC022687 protein (c14orf79).

The term “nucleotide sequence ENSGALG00000011687” as used herein refers to a polynucleotide sequence at nucleotides 54346493 to 54376693 on chicken Chromosome 5. The polynucleotide sequence encodes a polypeptide similar to vertebrate periaxin (PRX).

The term “nucleotide sequence ENSGALG00000011656” as used herein refers to a polynucleotide sequence at nucleotides 54336971 to 54344962 on chicken Chromosome 5. The polynucleotide sequence encodes the polypeptide AHNAK2 similar to KIAA2019.

The term “nucleotide sequence ENSGALG00000011646” as used herein refers to a polynucleotide sequence at nucleotides 54320538 to 54332563 on chicken Chromosome 5. The polynucleotide sequence encodes the polypeptide PLD4.

The term “nucleotide sequence ENSGALG00000011639” as used herein refers to a polynucleotide sequence at nucleotides 54263981 to 54313829 on chicken Chromosome 5. The polynucleotide sequence encodes a polypeptide similar to KIAA0284.

The term “nucleotide sequence ENSGALG00000023025” as used herein refers to a polynucleotide sequence at nucleotides 54222335 to 54223726 on chicken Chromosome 5. The polynucleotide sequence encodes a polypeptide.

The term “nucleotide sequence ENSGALG00000011618” as used herein refers to a polynucleotide sequence at nucleotides 54073641 to 54096083 on chicken Chromosome 5. The polynucleotide sequence encodes the polypeptide adenylosuccinate synthetase isozyme 1 (ADSS L1).

The term “nucleotide sequence ENSGALG00000011608” as used herein refers to a polynucleotide sequence at nucleotides 54024011 to 54038102 on chicken Chromosome 5. The polynucleotide sequence encodes the polypeptide inverted formin-2 (HBEBP2-binding protein C).

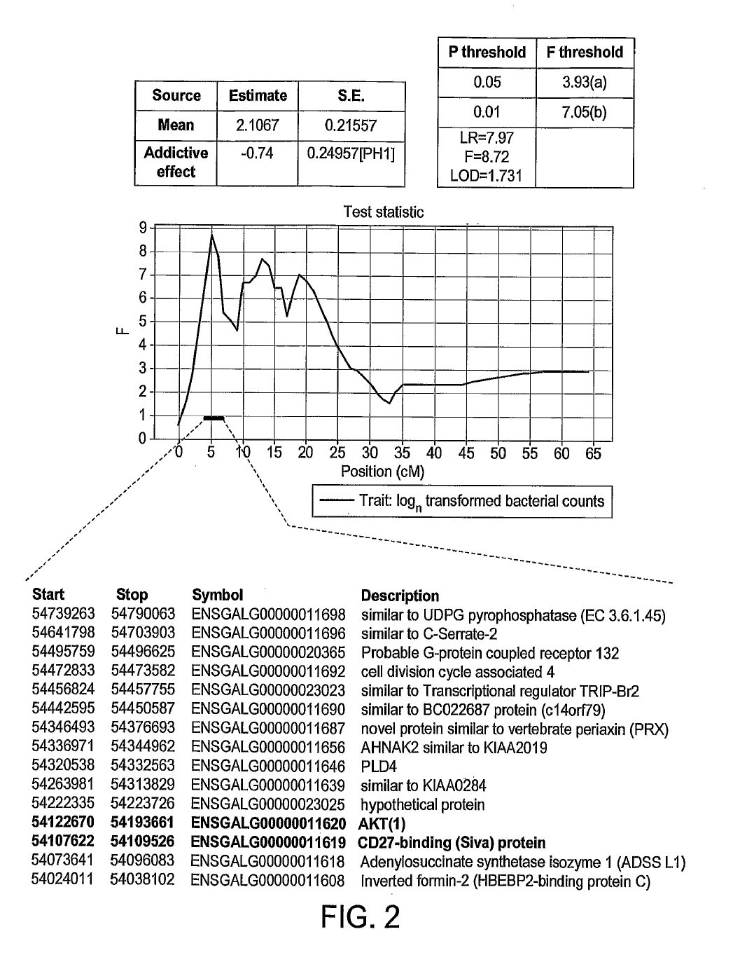

The numbering used herein (such as in Table A and FIG. 2) is based on the ENSEMBL release 50 for the chicken genome. FIG. 2 details the ENSGALG identifiers of candidate genes in the SAL1 locus.

### Allelic Variants

The phrase “determining that there is an allelic variant at a marker of the SAL1 locus” as used herein refers to the identification of the presence of two or more types of alleles at a marker which lies in the SAL1 locus.

The identification of allelic variants at a marker can be determined using recombinant DNA techniques such as PCR and DNA sequencing.

Association of Genotypes with Resistance/Susceptibility to Bacterial Infection

There are lines of inbred chickens which are either resistant to bacterial infection (such as Salmonella infection) or susceptible to bacterial infection (such as Salmonella infection).

Examples of inbred chicken lines resistant to Salmonella infection include the lines W1, 6₁ and N (Wigley, Hulme et al 2002; Microbes and Infection 4: 1111-1120). These birds can be obtained from the Poultry Production Unit, Institute for Animal Health, Compton, UK.

Examples of inbred chicken lines susceptible to Salmonella infection include the lines 7₂, C and 15I (Wigley, Hulme et al 2002; Microbes and Infection 4: 1111-1120). These birds can be obtained from the Poultry Production Unit, Institute for Animal Health, Compton, UK.

A genotype associated with resistance to bacterial infection (such as infection by Salmonella) can be determined, for example, by determining what the genotype is for a marker at the SAL1 locus in an animal of an inbred strain which is resistant to bacterial infection—this is the reference. Further, the genotype of more than one reference animal can be determined. Subsequently the genotypes of other animals at this marker can be compared with the reference or references and those animals with the same genotype as that of the reference can be identified. The comparison can be carried out on more than one marker at the SAL1 locus. In addition, an animal which has a genotype at one or more markers which is the same as that of the reference or references can be predicted as being resistant to infection by bacteria such as Salmonella. However, an animal which has a genotype at one or more markers which is different to that of the reference or references can be predicted as not being resistant to infection by bacteria such as Salmonella. The phrases “predict the response” and “predicting the response”, as used herein, refer to this type of comparison.

A genotype associated with susceptibility to bacterial infection (such as Salmonella) can be determined, for example, by determining what the genotype is for a marker at the SAL1 locus in an animal of an inbred strain which is susceptible to bacterial infection—this is the reference. The genotype of more than one reference animal can be determined. Subsequently the genotypes of other animals at this marker can be compared with the reference or references and those animals with the same genotype as that of the reference can be identified. The comparison can be carried out on more than one marker at the SAL1 locus. In addition, an animal which has a genotype at one or more markers which is the same as that of the reference or references can be predicted as being susceptible to infection by bacteria such as Salmonella. However, an animal which has a genotype at one or more markers which is different to that of the reference or references can be predicted as not being susceptible to infection by bacteria such as Salmonella. Again, the phrases “predict the response” and “predicting the response”, as used herein, refer to this type of comparison.

### Quantitative Trait Locus (QTL)

The resistance/susceptibility of an animal to bacterial infections, such as Salmonella, is a quantitative trait.

By using QTL analysis, as described in Example 1, the present inventors have associated resistance/susceptibility to bacterial infection with the region 54.0 MB to 54.8 MB on chicken Chromosome 5 (i.e. the SAL1 locus).

As used herein, the terms “quantitative trait locus” (QTL) refers to an association between a marker and a chromosomal region that affects the phenotype of a trait of interest—which in the present case is resistance/susceptibility to bacterial infection. Typically, the association is determined statistically; e.g., based on one or more methods published in the literature (see, for example, Zeng et al 1994 Genetics, Vol 136, 1457-1468; Sen and Churchill, 2001 Genetics, Vol. 159, 371-387). A QTL can be a chromosomal region and/or a genetic locus with at least two alleles that differentially affect the expression of the phenotypic trait of interest.

### Animals

In one example, the animal mentioned herein is a non-human animal.

The animal may be a bird such as a domestic fowl or a gallinaceous bird. Examples of domestic fowl include turkeys, chickens, ducks, guinea fowl, quail and geese.

In one example, the animal may be a chicken (Gallus gallus).

The sample for use herein may be a blood sample.

The sample for use herein may be a genomic DNA preparation—such as genomic DNA derived (derivable) from a blood sample.

### Animals Resistant to Bacterial Infection

Animals which are resistant to bacterial infection or which have an increased resistance to bacterial infection can be produced by selective breeding programmes or by genetic engineering and by the breeding of the transgenic animals.

In one example, the animal is in the form of a fertilised egg when, for instance, the animal is a fowl.

As used herein, the phrase “an animal which is resistant to bacterial infection” refers to an animal which has a genotype associated with resistance to bacterial infection (such as Salmonella infection) at one or more markers of the SAL1 locus.

As used herein, the phrase “increasing the resistance to bacterial infection” refers to method in which at least part of a SAL1 locus having a genotype associated with resistance to bacterial infection at one or more markers is replaced with at least part of a corresponding SAL1 locus having a genotype which is associated with a stronger resistance to bacterial infection (such as Salmonella infection).

By comparing the resistance to bacterial infections of (i) animals having one type of genotype associated with resistance to bacterial infection at one or more markers of the SAL1 locus with (ii) animals having a different type of genotype associated with resistance to bacterial infection at one or more markers of the SAL1 locus, genotypes at a marker and/or combinations genotypes at several markers can be identified which have a stronger (i.e. better) resistance to bacterial infection than others.

In selective breeding programmes to produce animals which are resistant to bacterial infection, at least one animal with a genotype associated with resistance to bacterial infection at one or more markers of the SAL1 locus are identified, selected and used for breeding. Offspring of such a cross are then identified which have a genotype associated with resistance to bacterial infection at one or more markers of the SAL1 locus. These offspring may then be used for selective breeding. Again, the offspring of a breeding pair in a selective breeding programme are subject to selection by determining if they have a genotype associated with resistance to bacterial infection at one or more markers of the SAL1 locus. Many rounds of selective breeding may be carried out using animals with a genotype associated with resistance to bacterial infection at one or more markers of the SAL1 locus.

In any one breeding pair, each animal may be derived from a different genetic background (strain or line) in order to, for example, minimise the occurrence of undesirable genetic disorders (such as recessive disorders) and to maximise genetic diversity.

In selective breeding programmes to produce animals which have an increased resistance to bacterial infection, at least one animal with a genotype associated with stronger resistance to bacterial infection at one or more markers of the SAL1 locus are identified, selected and used for breeding. Offspring of such a cross are then identified which have a genotype associated with stronger resistance to bacterial infection at one or more markers of the SAL1 locus. These offspring may then be used for selective breeding. Again, the offspring of a breeding pair in a selective breeding programme are subject to selection by determining if they have a genotype associated with stronger resistance to bacterial infection at one or more markers of the SAL1 locus. Many rounds of selective breeding may be carried out using animals with a genotype associated with stronger resistance to bacterial infection at one or more markers of the SAL1 locus.

In any one breeding pair, each animal may be derived from a different genetic background (strain or line) in order to minimise the occurrence of undesirable genetic disorders and to maximise genetic diversity.

The selective breeding programme uses conventional breeding techniques. However, in addition, in order to identify suitable resistant/susceptible animals the genotype of one or more markers at the SAL1 locus (which lies between 54.0 MB to 54.8 MB of chicken Chromosome 5 or an equivalent thereof) is determined.

In one aspect, the present invention relates to the use of one or more markers at the SAL1 locus in a selective breeding programme for producing an animal which is resistant to bacterial infection or increasing the resistance to bacterial infection, wherein the SAL1 locus lies between 54.0 MB to 54.8 MB of chicken Chromosome 5 or an equivalent thereof.

In a further aspect, the present invention relates to the use of one or more markers at the SAL1 locus in a selective breeding programme for producing an animal which is susceptible to bacterial infection or increasing the susceptibility to bacterial infection, wherein the SAL1 locus lies between 54.0 MB to 54.8 MB of chicken Chromosome 5 or an equivalent thereof.

As an alternative to selective breeding, animals which are resistant to bacterial infection or which have an increased resistance to bacterial infection can be produced by genetic engineering methods. Such genetic engineering methods comprise the step of replacing at least part of the SAL1 locus with a SAL1 locus or corresponding part thereof from an animal which is resistant to bacterial infection.

The term “part of the SAL1 locus” may comprise, for example, one, two or three markers of the SAL1 locus.

The phrase “a SAL1 locus or corresponding part thereof from an animal which is resistant to bacterial infection” as used herein refers to a SAL1 locus which is derived or derivable from an animal which has a genotype associated with resistance to bacterial infections (such as Salmonella) at one or more markers.

Vectors for use in the methods described herein comprise at least part of the SAL1 locus from an animal which is resistant to bacterial infection.

The replacement of ‘at least part of the SAL1 locus’ with ‘a SAL1 locus or corresponding part thereof from an animal which is resistant to bacterial infection’ may occur by homologous recombination.

The introduction into an animal cell of a vector comprising at least part of the SAL1 locus may be accomplished by any available technique, including transformation/transfection, delivery by viral or non-viral vectors and microinjection. Each of these techniques is known in the art. A useful general textbook on Techniques for producing transgenic animals is Houdebine, Transgenic animals—Generation and Use (Harwood Academic, 1997)—which is an extensive review of the techniques used to generate transgenic animals.

Technologies for embryo micromanipulation now permit the introduction of suitable vectors into, for example, fertilised ova. For instance, totipotent or pluripotent stem cells can be transformed by microinjection, calcium phosphate mediated precipitation, liposome fusion, retroviral infection or other means, the transformed cells are then introduced into the embryo, and the embryo then develops into a transgenic animal. In one example, developing embryos are infected with a retroviral vector containing the replacement DNA (for instance, the vector contains at least part of a SAL1 locus from an animal which is resistant to bacterial infection), and transgenic animals produced from the infected embryo. In another example, the appropriate vector or vectors are co-injected into the pronucleus or cytoplasm of embryos, preferably at the single cell stage, and the embryos allowed to develop into mature transgenic animals. These techniques as well known (see reviews of standard laboratory procedures for microinjection of DNA into mammalian fertilised ova, including Hogan et al., Manipulating the Mouse Embryo, (Cold Spring Harbor Press 1986); Krimpenfort et al., Bio/Technology 9:844 (1991); Palmiter et al., Cell, 41: 343 (1985); Kraemer et al., Genetic manipulation of the Mammalian Embryo, (Cold Spring Harbor Laboratory Press 1985); Hammer et al., Nature, 315: 680 (1985); Wagner et al., U.S. Pat. No. 5,175,385; Krimpenfort et al., U.S. Pat. No. 5,175,384, the respective contents of which are incorporated herein by reference). Injected eggs are then cultured before transfer into the oviducts of pseudopregnant recipients.

Analysis of animals which may contain transgenic sequences would typically be performed by either PCR or Southern blot analysis following standard methods. If desired, the organism can be bred to homozygosity.

In one example, a transgenic bird (such as a chicken) may be produced by a method comprising infecting a bird egg with a vector comprising at least part of the SAL1 locus from an animal which is resistant to bacterial infection. For instance, the embryonic blastodisc of the bird egg is contacted with the vector. In more detail, transgenic birds are generated by delivering a vector to the primordial germ cells of early stage avian embryos. For instance, freshly laid eggs are obtained and placed in a temperature controlled, humidified incubator. The embryonic blastodisc in the egg is gradually rotated to lie on top of the yolk. This may be accomplished by any method known in the art, such as by rocking the egg regularly. The vector is subsequently delivered into the space between the embryonic disk and the perivitelline membrane; although the vector may be delivered by any known method. In one example, a window is opened in the shell, the vector is injected through the window and the shell window is closed. The eggs may then be incubated until hatching. Hatched chicks may be raised to sexual maturity and mated.

In one example, transgenic mammals may also be produced by nuclear transfer technology as described in Schnieke, A. E. et al., 1997, Science, 278: 2130 and Cibelli, J. B. et al., 1998, Science, 280: 1256. Using this method, fibroblasts from donor mammals are stably transfected with a vector incorporating the sequences of interest (such as a vector comprising at least part of the SAL1 locus from a mammal which is resistant to bacterial infection). Stable transfectants are then fused to enucleated oocytes, cultured and transferred into female recipients.

By way of a specific example for the construction of transgenic mammals, vectors (such as a vector comprising at least part of the SAL1 locus from an animal which is resistant to bacterial infection) are microinjected using, for example, the technique described in U.S. Pat. No. 4,873,191, into oocytes which are obtained from ovaries freshly removed from the animal. The oocytes are aspirated from the follicles and allowed to settle before fertilisation with thawed frozen sperm capacitated with heparin and prefractionated by Percoll gradient to isolate the motile fraction.

The fertilised oocytes are centrifuged, for example, for eight minutes at 15,000 g to visualise the pronuclei for injection and then cultured from the zygote to morula or blastocyst stage in oviduct tissue-conditioned medium. This medium is prepared by using luminal tissues scraped from oviducts and diluted in culture medium. The zygotes must be placed in the culture medium within two hours following microinjection.

Oestrous is then synchronized in the intended recipient mammals by administering coprostanol. Oestrous is produced within two days and the embryos are transferred to the recipients 5-7 days after oestrous. Successful transfer can be evaluated in the offspring by Southern blot.

Alternatively, the vectors (such as a vector comprising at least part of the SAL1 locus from an animal which is resistant to bacterial infection) can be introduced into embryonic stem cells (ES cells) and the cells cultured to ensure modification by the transgene. The modified cells are then injected into the blastula embryonic stage and the blastulas replaced into pseudopregnant hosts. The resulting offspring are chimeric with respect to the ES and host cells, and nonchimeric strains which exclusively comprise the ES progeny can be obtained using conventional cross-breeding. This technique is described, for example, in WO91/10741.

The vectors which may be used in the methods mentioned herein include viral vectors, such as adenoviral vectors, retroviral vectors, baculoviral vectors and herpesviral vectors. Such techniques have moreover been described in the art, for example by Zhang et al. (Nucl. Ac. Res., 1998, 26:3687-3693).

In one example, a lentiviral vector such as an equine infectious anaemia virus (EIAV) vector, is used to produce transgenic birds such as chickens. The use of lentiviral vectors to produce transgenic avians may allow the expression of genes throughout significant numbers of generations without the foreign gene silencing observed with some retroviral vectors.

Vectors comprising at least part of the SAL1 locus from an animal which is resistant to bacterial infection may be used to transduce cells in the blastoderm stage embryo in new-laid eggs by injection. Alternatively, vectors can be used to transduce earlier stage embryos using techniques such as those described in WO 90/13626 or similar published techniques to allow the embryo to develop normally.

In brief, a uterine embryo is abstracted from a hen either manually or by inducing premature oviposition. The embryo is transduced with the lentiviral vector and then cultured to fruition. This allows cells of the embryo to be transduced whilst the number of cells present is relatively low and increases the number of birds produced in which the introduced gene is present in the germ line and is inherited.

Construction of vectors for use in methods of the invention may employ conventional ligation techniques. Isolated viral vectors, plasmids or DNA fragments are cleaved, tailored, and religated in the form desired to generate the plasmids required. If desired, analysis to confirm correct sequences in the constructed vectors is performed in a known fashion such as by Southern blotting, dot blotting, PCR or in situ hybridisation, using an appropriately labelled probe. Those skilled in the art will readily envisage how these methods may be modified, if desired. Vectors useful in the present invention are advantageously provided with marker genes to facilitate identification and localisation.

### Kits

Kits for identifying in a sample the genotype of one or more markers at the SAL1 locus comprise a means for determining alleles of one or more markers.

The term “means for determining alleles” as used herein refers to any means by which the different alleles of a marker at the SAL1 locus can be identified. For instance, the means for determining alleles of a marker is at least one oliognucleotide primer or oliognucleotide probe. Examples of such means include oligonucleotide primers or probes which are specific for SNPs. Examples of other means include oligonucleotide primers or probes which are specific for microsatellite markers.

A kit according to the present invention is one comprising the means for determining the alleles of one or more markers selected from the group consisting of:
the single nucleotide polymorphism SNP2 (rs16511470) on chicken Chromosome 5;
the microsatellite marker ADL166 (UniSTS:71823) on chicken Chromosome 5;
a polymorphism in the nucleotide sequence ENSGALG00000011620 (AKT1) on chicken Chromosome 5; and
a polymorphism in the nucleotide sequence ENSGALG00000011619 (SIVA1) on chicken Chromosome 5.

Using techniques known in the art, oligonucleotide primers and oligonucleotide probes for each allele of each marker can be produced.

Examples of a kit according to the present invention include ones comprising the means for determining the alleles of the single nucleotide polymorphism SNP2 (rs16511470) on chicken Chromosome 5 and/or the microsatellite marker ADL166 (UniSTS:71823) on chicken Chromosome 5.

Examples of the means for determining alleles of a marker wherein said marker is a single nucleotide polymorphism SNP2 (rs16511470) on chicken Chromosome 5, include oliognucleotide primers having the sequence 5′-ATCTCAGCCCCATAAAAACGC-3′ (SEQ ID NO 44), 5′-TAGAGTCGGGGTATTTTTGCG-3′ (SEQ ID NO 45), 5′-ATCTCAGCCCCATAAAAACGT-3′ (SEQ ID NO 46) and 5′-TAGAGTCGGGGTATTTTTGCA-3′ (SEQ ID NO 47).

An example of a means for determining alleles of a marker wherein said marker is the microsatellite marker ADL166 (UniSTS:71823) on chicken Chromosome 5, is oliognucleotide primers pairs or oliognucleotide probes having the sequence 5′-TGCCAGCCCGTAATCATAGG-3′ (SEQ ID NO 40) and 5′-AAGCACCACGACCCAATCTA-3′ (SEQ ID NO 41).

A further example of a means for determining alleles of a marker wherein said marker is the microsatellite marker ADL166 (UniSTS:71823) on chicken Chromosome 5, is oliognucleotide primers pairs or oliognucleotide probes having the sequence 5′-ACGGTCGGGCATTAGTATCC-3′ (SEQ ID NO 48) and 5′-TTCGTGGTGCTGGGTTAGAT-3′ (SEQ ID NO 49).

A kit as described herein may further comprise instructions for identifying the genotype of said one or more markers.

### Arrays

The term “array” as used herein refers to oligonucleotide primers or oligonucleotide probes which have been fixed or immobilised, in a systematic order, onto a solid substrate.

Array technology and the various techniques and applications associated with it is described generally in numerous textbooks and documents. Array technology used in SNP analysis is discussed in Wang et al., 1998, Science 280(5366):1077-82.

One example of DNA arrays is an array of oligonucleotide (˜20-˜25-mer oligos) probes synthesized either in situ (on-chip) or by conventional synthesis followed by on-chip immobilization. The array is exposed to labelled sample DNA, hybridized, and the identity of complementary sequences are determined. Such a DNA chip is sold by Affymetrix, Inc., under the GeneChip® trademark.

Examples of some commercially available microarray formats are set out, for example, in Marshall and Hodgson, 1998, Nature Biotechnology 16(1):27-31.

The invention will now be further described by way of Examples, which are meant to serve to assist one of ordinary skill in the art in carrying out the invention and are not intended in any way to limit the scope of the invention.

### EXAMPLES

The present invention employs, unless otherwise indicated, conventional techniques of chemistry, molecular biology, microbiology, recombinant DNA and immunology, which are within the capabilities of a person of ordinary skill in the art.

### Example 1

### Materials and Methods:

### Animals

Line 6₁ (resistant) and 15I (susceptible) parental birds were selected for their divergent phenotypes of susceptibility to systemic salmonellosis. Parent lines were maintained under specific pathogen-free conditions and tested to be free of salmonella. To generate the backcross (BC1), the F1 progeny were crossed onto the susceptible line 15I parent stock. The BC1 population was used in the original mapping of the Salmonellosis QTL in which SAL1 was initially identified (Mariani, Barrow et al. 2001). All subsequent generations were produced by backcrossing the progeny of each backcross generation onto the susceptible 15I parent line. Each generation was screened with microsatellite markers flanking SAL1 (ADL166, COM184, ADL233, MCW81, MCW29). Only birds carrying line 6₁ alleles for these markers were retained as parents for the next generation of congenic backcross birds. An advanced backcross (BC6) mapping population was used in this study.

### Bacterial Challenge

All chicks were reared under disease-free conditions until two weeks of age. To assess the level of susceptibility of the backcross progeny, two-week-old chicks were intravenously challenged with 10⁵ S. enterica serovar Typhimurium F98. At 5 days post-infection (dpi) birds were killed and the spleen aseptically removed and weighed. To assess the level of bacterial colonisation spleens were weighed and homogenized in phosphate buffered saline. A series of 10-fold dilutions were performed on the homogenate and 100 μl plated onto MacConkey Agar supplemented with 20 μg/ml nalidixic acid. Bacterial counts were measured as total bacterial counts per spleen.

### DNA Extractions

Genomic DNA was extracted from whole blood as previously described (Bumstead, Messer et al. 1987).

### Microsatellite Genotyping

Microsatellite markers (ADL166, MCW081, MCW029) were selected for polymorphic divergence between the parent lines. PCR amplification was carried out using 100 ng genomic DNA, 200 μM of each dNTP, 0.25 pmol of each primer in a total reaction volume of 10 μl. One primer of each pair was fluorescently labelled for detection during fragment analysis. Cycling conditions were as follows: 94° C. for 4 min; 30 cycles of 94° C. for 1 min, 50-60° C. (assay dependant) for 1 min and 72° C. for 2 mins. Products were run out on a Beckman CEQ8000 capillary sequencer using size standard 600. Genotypes were assessed using the Beckman CEQ8000 software for fragment analysis.

### SNP Detection and Genotyping

121 SNPs flanking the existing SAL1 locus (36 428 188-56 139 321 bp based on Gallus gallus genome Build 2.1 release 50) were screened in the parent lines to identify fully informative markers for the mapping study, using previously identified SNPs available through ENSEMBL and existing panels of chicken SNPs available on the Illumina BeadStation genotyping platform. SNPs were selected on the basis of their homozygosity and the divergence of the homozygous allele in the parent lines. Thus, only 37 SNPs (see Table A) that were fully fixed and divergent between the parent lines were selected for a fully informative analysis. Informative SNPs were PCR amplified using 50-100 ng genomic DNA, 200 μM of each dNTP, 400 pM of each primer in a total reaction volume of 12.5 μl and genotyped in the backcross mapping panel using a fragment analysis assay on the Beckman CEQ8000 capillary sequencer. Cycling conditions using “touchdown PCR”, were as follows: 95° C. for 2 mins, 30 secs denaturing, 30 secs of annealing starting at 5° C. above calculated annealing temp and dropping by 1° C. in each cycle, and 2 min extension at 72° C. A further 25 cycles were performed at the annealing temp, followed by a final cycle of 4 min extension at 72° C. PCR products were purified by incubating with ExoSAP-IT (Amersham) for 45 mins followed by enzyme inactivation at 80° C. for 15 min. Multiplexing of 2-5 PCR products in a single reaction used 1 μl of each product. SNP assay reaction was carried out as follows: (3.5 μl) of cleaned-up product were combined with 4 μl SNPStart mastermix (Beckman) and 50 pM each SNP assay primer in a 10 μl reaction. Each assay primer in a given multiplex was designed to be of a different length for accurate genotyping during fragment analysis.

Table A details the sequence of primers used in genotyping assays. Where applicable the universal identifier (rsSNP number or UniSTS number) is used for previously validated SNPs. The Chromosome position of those markers without universal identifiers (no dbSNP) is based on the numbering used in ENSEMBL release 50 for the chicken genome (Gallus gallus genome Build 2.1). The prefix “a” refers to the assay SNP. Microsatellites mentioned herein are described in Mariani et al 2001. SNPs mentioned herein may have been used in the study described in Wong et al 2004.

TABLE A

| | | | Chromosome |
|---|---|---|---|
| | | Universal | 5 position |
| Marker | Assay Primer sequence | identifier | (bp) |
| aSNP163 | TTTTTCATATTTTATTGTAACAAAC[C/A] | rs15697509 | |
| aSNP169 | CCTAGTGTAAGTGGCACCCAATCCGTCCACCTGTG[G/A] | rs13586706 | |
| aSNP154 | CTGCTCCTGTAAAGTGACCTGTTCCTGAAG[T/C] | rs14536896 | |
| aSNP134 | TACAGTGTTACAATTAAAAACTGAT[A/G] | rs14537044 | |
| aSNP153 | CCAAACCAGATTAGTATGTAGTTAG[T/C] | rs14537875 | |
| aSNP132 | AATAAGAACCTCAGAGCTCTTTAAATAATTTCTCACAATG[T/C] | no dbSNP | 43,668,563 |
| aSNP131 | CTGACCGGTTTAGTACTACTCCCGTATCACTATTAGTAAC[G/T] | rs16498139 | |
| aSNP130 | TTATATTAAGTGTAAAATAAGATGTCTTTTATTCA[T/C] | rs13587759 | |
| MCW0029 | CATGCAATTCAGGACCGTGCA/GTGGACACCCATTTGTACCCTATG | UniSTS: 280083 | |
| aSNP126 | GTTCCAGAGAAATGGAATTATATTTGTTTT[C/A] | rs13587880 | |
| aSNP152 | GATGTATTTGTGTAGAACTC[G/A] | rs16498806 | |
| aSNP124 | GCTAAAGATAGAAAAAGCAAATTCT[G/A] | rs16499027 | |
| aSNP151 | AAGTCAGTCACTATAATAAAGGCAAATCTTCCCAG[G/A] | rs15711698 | |
| aSNP139 | TCTGTAGGAAAGGAATCTGTATGGAAA[A/G] | rs14538974 | |
| aSNP142 | GCTCTGTGCTTAAAAGGATACTCTGATTTGGAA[T/C] | rs15712602 | |
| aSNP119 | TTAAGCTCTCTGAGTGTACTTTTA[A/T] | no dbSNP | 46,110,966 |
| aSNP38 | CAGGACGTATCGACAGGAAAATAGAGTTCCC[G/T] | rs10724280 | |
| aSNP36 | ACCTGGTCATGATAAACTTCATGCAACTTCACTAC[G/A] | rs16501189 | |
| aSNP29 | GGGTCCCCCTCCCAGAGGCTTTGGGTGGCC[T/C] | no dbSNP | 47,841,841 |
| aSNP28 | GAGAGAAGAAAATGTTCTTATTAGG[T/C] | no dbSNP | 48,004,531 |
| aSNP26 | GTTCATCATCAAACAGTGCA[A/G] | rs16503833 | |
| aSNP21 | ATGTGGATCCAGATGATGACGAGGTGGTGGGCTGAATAGTGGAGG[T/C] | rs15722361 | |
| aSNP74 | TAGACAGCAGTAAGTGACAAAGTATCTGCCTAAATCAGCT[G/A] | no dbSNP | 50,873,575 |
| aSNP16 | CAGGAAAGAAGCTTCCTCCTGGTGGAATACCTGGCATTGA[T/C] | rs14545353 | |
| aSNP76 | GAGAACAAAATGTTTCAAATGTTGTCAGAGTAGACCTGGAGTA[G/A] | no dbSNP | 51,068,822 |
| MCW0081 | GTTGCTGAGAGCCTGGTGCAG/CCTGTATGTGGAATTACTTCTC | UniSTS: 280117 | |
| aSNP81 | CATGCTCTAGCCCTTAATATTTTCAAATGTTAGTC[T/C] | no dbSNP | 51,572,567 |
| aSNP83 | ATGGCTGTTCAAAAGTAACC[G/A] | rs14546163 | |
| aSNP85 | GCATCCTCCAAAGCCATTGC[T/C] | rs14546524 | |
| aSNP91 | ATTCTGTGATTCTATGATTCTAACA[G/A] | no dbSNP | 52,600,248 |
| aSNP92 | TCAGATAAACTCTTGCATAGTTTCTCAGTTGATTTAGCTCCTTATCTC[G/A] | no dbSNP | 52,704,194 |
| aSNP7 | AGCCGAAGCCTCTTGAGGACTTCTCCAAACTTCTC[G/A] | rs16509559 | |
| aSNP94 | TGATTAAGTGCTACTAAGTATCATACACCTTATGATTTGC[T/C] | rs16509651 | |
| aSNP5 | TTCTACTTCTTTCTTGCAAAAATAAACTCA[T/A] | no dbSNP | 53,108,576 |
| aSNP96 | AGTTTGTGAGCATACTGTTACTCTTTAGATTTCAT[T/C] | no dbSNP | 53,111,114 |
| aSNP4 | AATATGTACATCATGAGAGCTTGAC[G/A] | no dbSNP | 53,310,375 |
| aSNP2 | ATCTCAGCCCCATAAAAACG[C/T] | rs16511470 | |
| ADL0166 | TGCCAGCCCGTAATCATAGG/AAGCACCACGACCCAATCTA | UniSTS: 71823 | |
| aSNP170 | CTGCTTTCTGCTCTCGAGTT[G/A] | rs16513188 | |
| aSNP171 | TGTATGCCAACACCAACCGATACCA[C/T] | rs14551368 | |

TABLE B
the SEQ ID Nos of the primers detailed in Table A.

| | | SEQ |
|---|---|---|
| Marker | Assay Primer sequence | ID No |
| aSNP163 | TTTTTCATATTTTATTGTAACAAAC[C/A] | 1 |
| aSNP169 | CCTAGTGTAAGTGGCACCCAATCCGTCCACCTGTG[G/A] | 2 |
| aSNP154 | CTGCTCCTGTAAAGTGACCTGTTCCTGAAG[T/C] | 3 |
| aSNP134 | TACAGTGTTACAATTAAAAACTGAT[A/G] | 4 |
| aSNP153 | CCAAACCAGATTAGTATGTAGTTAG[T/C] | 5 |
| aSNP132 | AATAAGAACCTCAGAGCTCTTTAAATAATTTCTCACAATG[T/C] | 6 |
| aSNP131 | CTGACCGGTTTAGTACTACTCCCGTATCACTATTAGTAAC[G/T] | 7 |
| aSNP130 | TTATATTAAGTGTAAAATAAGATGTCTTTTATTCA[T/C] | 8 |
| MCW0029 | CATGCAATTCAGGACCGTGCA | 9 |
| | GTGGACACCCATTTGTACCCTATG | 10 |
| aSNP126 | GTTCCAGAGAAATGGAATTATATTTGTTTT[C/A] | 11 |
| aSNP152 | GATGTATTTGTGTAGAACTC[G/A] | 12 |
| aSNP124 | GCTAAAGATAGAAAAAGCAAATTCT[G/A] | 13 |
| aSNP151 | AAGTCAGTCACTATAATAAAGGCAAATCTTCCCAG[G/A] | 14 |
| aSNP139 | TCTGTAGGAAAGGAATCTGTATGGAAA[A/G] | 15 |
| aSNP142 | GCTCTGTGCTTAAAAGGATACTCTGATTTGGAA[T/C] | 16 |
| aSNP119 | TTAAGCTCTCTGAGTGTACTTTTA[A/T] | 17 |
| aSNP38 | CAGGACGTATCGACAGGAAAATAGAGTTCCC[G/T] | 18 |
| aSNP36 | ACCTGGTCATGATAAACTTCATGCAACTTCACTAC[G/A] | 19 |
| aSNP29 | GGGTCCCCCTCCCAGAGGCTTTGGGTGGCC[T/C] | 20 |
| aSNP28 | GAGAGAAGAAAATGTTCTTATTAGG[T/C] | 21 |
| aSNP26 | GTTCATCATCAAACAGTGCA[A/G] | 22 |
| aSNP21 | ATGTGGATCCAGATGATGACGAGGTGGTGGGCTGAATAGTGGAGG[T/C] | 23 |
| aSNP74 | TAGACAGCAGTAAGTGACAAAGTATCTGCCTAAATCAGCT[G/A] | 24 |
| aSNP16 | CAGGAAAGAAGCTTCCTCCTGGTGGAATACCTGGCATTGA[T/C] | 25 |
| aSNP76 | GAGAACAAAATGTTTCAAATGTTGTCAGAGTAGACCTGGAGTA[G/A] | 26 |
| MCW0081 | GTTGCTGAGAGCCTGGTGCAG | 27 |
| | CCTGTATGTGGAATTACTTCTC | 28 |
| aSNP81 | CATGCTCTAGCCCTTAATATTTTCAAATGTTAGTC[T/C] | 29 |
| aSNP83 | ATGGCTGTTCAAAAGTAACC[G/A] | 30 |
| aSNP85 | GCATCCTCCAAAGCCATTGC[T/C] | 31 |
| aSNP91 | ATTCTGTGATTCTATGATTCTAACA[G/A] | 32 |
| aSNP92 | TCAGATAAACTCTTGCATAGTTTCTCAGTTGATTTAGCTCCTTATCTC[G/A] | 33 |
| aSNP7 | AGCCGAAGCCTCTTGAGGACTTCTCCAAACTTCTC[G/A] | 34 |
| aSNP94 | TGATTAAGTGCTACTAAGTATCATACACCTTATGATTTGC[T/C] | 35 |
| aSNP5 | TTCTACTTCTTTCTTGCAAAAATAAACTCA[T/A] | 36 |
| aSNP96 | AGTTTGTGAGCATACTGTTACTCTTTAGATTTCAT[T/C] | 37 |
| aSNP4 | AATATGTACATCATGAGAGCTTGAC[G/A] | 38 |
| aSNP2 | ATCTCAGCCCCATAAAAACG[C/T] | 39 |
| ADL0166 | TGCCAGCCCGTAATCATAGG | 40 |
| | AAGCACCACGACCCAATCTA | 41 |
| aSNP170 | CTGCTTTCTGCTCTCGAGTT[G/A] | 42 |
| aSNP171 | TGTATGCCAACACCAACCGATACCA[C/T] | 43 |

### Statistical Analysis and QTL Mapping

Genomic locations were based on the published sequence of the chicken (Gallus gallus) genome (Build 2.1) www.ensembl.org. QTL analysis was performed by regression interval mapping (Haley and Knott 1992) using QTL Express software (Seaton, Haley et al. 2002) available through GRIDQTL (http://gridqt1.cap.ed.ac.uk). This approach is based on the regression of phenotypes on probabilities of inheriting the QTL at the position being tested. QTL Express assumes that the distribution of the phenotype is normal. Since the bacterial counts were not normally distributed a logarithmic transformation was applied (ln). Permutation analysis (n=1000 cycles) was used to set significance levels for the trait under investigation. To allow for possible differences in spleen size resulting from genetic differences or differential levels of infiltration due to infection, the bacterial counts were also investigated using spleen weight as a covariate in the analysis using gridQTL (Seaton, Haley et al. 2002).

### Results:

Means and standard errors of the raw data for Line 6₁ (resistant) and 15I (susceptible) parental birds are given in Table 1. The coefficient of variation of different parent line groups was high (16 to 79% for spleen count). A one-way ANOVA was conducted on the raw data (Table 1). Spleen counts were transformed to natural logarithms to account for the non-normal distribution of the data and means and back-transformed means are presented in Table 2. There was a significant (P=0.01) difference between the groups with the F1 and backcross showing intermediate mean values. An additive effect of 0.74 was calculated for the QTL that explains a substantial proportion of the variation between the parent lines: the line difference from Table 1 is 1.52 and twice the additive effect of the QTL is 1.48 ln spleen count.

TABLE 1
Means (backtransformed) and standard errors for spleen count of
bacteria per ml (×10⁶) for the parental lines 6₁ and 15I and the
F1 and Backcross generations.

| | | Spleen count (N) × 10⁶ | |
|---|---|---|---|
| Genotype | Number | Mean | se |
| Line 6₁ | 4 | 5.62 | 0.91 |
| Line 15I | 5 | 24.8 | 1.93 |
| 15I × 6₁ F1 | 8 | 13.7 | 3.35 |
| 15I × (15I × 6₁) BC | 52 | 19.2 | 2.10 |

TABLE 2
Least squares means (backtransformed) and pooled standard
errors of differences (sed) for the natural logarithm of spleen
count of bacteria per ml (×10⁶) for the parental lines 6₁ and
15I and the F1 and Backcross generations.

| Genotype | In Spleen count (N) |
|---|---|
| Line 6₁ | 1.68 (5.4) |
| Line 15I | 3.20 (24.5) |
| 15I × 6₁ F1 | 2.48 (11.9) |
| 15I × (15I × 6₁) BC | 2.65 (14.1) |
| Pooled sed | 0.357 |
| Significance | P = 0.010 |

### Interval Mapping:

Analysis of the salmonellosis resistance QTL in the BC6 mapping population by interval mapping revealed a significant association with logₙ transformed bacterial counts in the spleen over a narrow range within the previously defined SAL1 region on Chromosome (Chr) 5. There were no differences in the results from the analysis of spleen counts with or without a covariate for spleen weight. Using a 1 LOD drop to estimate the confidence interval for the significant peak (LOD 1.73) refined the linkage peak further. The QTL location using this analysis indicates the significant region now extends from marker ADL0166 to SNP85 along the SAL1 locus (54.0-54.8 MB along Chr 5) (FIG. 2). There are fifteen genes encoded in the genome between these two markers.

### Discussion

In order to fine map the SAL1 locus the present inventors generated a congenic line carrying the QTL interval from the resistant line 6₁ on a homogenous background of the susceptible line 15I. The generation of these congenic lines allows assessment of the effect of the SAL1 QTL on the disease resistance phenotype. In mice, this approach has proven successful in the genetic dissection of many complex traits, including diseases such as epilepsy (Legare, Bartlett et al. 2000), obesity (Lembertas, Perusse et al. 1997), atherosclerosis (Wang, Shi et al. 2007) and type 1 diabetes (Todd 1999).

Using a panel of 40 markers (37 SNPs and 3 microsatellites—MS)—detailed in Table A)—the SAL1 locus has now been resolved to a small number of potential candidate genes which can be examined for possible functional effects resulting in the observed differential level of disease resistance. Differences in the pathology of infection between the resistant and susceptible lines indicate that the key to the resistance lies with mononuclear/phagocytic cell function (Wigley, Hulme et al. 2002). Salmonella typhimurium invades the host macrophages and can induce either an almost immediate cell death or establish an intracellular niche within the phagocytic vacuole (Monack, Navarre et al. 2001). Macrophages from adult birds of the resistant line cleared salmonella infections within 24 hrs whereas the susceptible line showed persistent infection beyond 48 h post infection (Wigley, Hulme et al. 2002). Clearance in line 6₁ birds was associated with the ability to limit the replication of the bacteria in the early stages of infection within the macrophage (Bumstead and Barrow 1993), suggesting a possible role for the functional gene in bacterial clearance and resistance.

The locus on chicken Chr 5 has conserved synteny with Human Chr 14 and highlights a number of potential candidate genes that may contribute to the observed differential phenotype in the parental lines.

By far the most salient of the candidates identified in the refined SAL1 locus are the apoptosis regulatory protein, Siva-1—CD27-binding protein—(encoded by the nucleotide sequence ENSGALG00000011619, and the signalling molecule RAC-alpha serine/threonine-protein kinase (AKT1) also known as protein kinase B (PKB) (encoded by the nucleotide sequence ENSGALG00000011620). Siva-1 is an apoptosis-inducing factor and a member of the tumour necrosis factor receptor (TFNR) superfamily (Yoon, Ao et al. 1999; Gudi, Barkinge et al. 2006). Apoptosis serves an essential role in the removal of infected cells and clearance of intracellular pathogens. Another member of the TNF superfamily, TRAIL (TNF-related apoptosis-inducing ligand) was identified by Malek & Lamont (2003) as a potential candidate gene for resistance to Salmonella enteritidis using a single SNP candidate gene approach in the chicken. The analysis showed TRAIL had associations with both spleen and caecal bacterial load (Malek and Lamont 2003) demonstrating a plausible role for the TNF-driven apoptosis pathway in salmonella infection.

The second candidate gene that we have identified in this study, AKT1, has also been implicated in clearance of salmonella. Central to pathogen survival is the intricate relationship between the host and bacterial proteins. Upon infection, the S. typhimurium effector protein SopB activates AKT1 in HeLa and IEC (rat small intestine epithelial) cells (Knodler, Finlay et al. 2005), promoting the intracellular survival of the bacteria by manipulating actin dynamics and phagosome-lysosome fusion (Kuijl, Savage et al. 2007). S. typhimurium modulates the kinesin motors on phagosomes, inhibiting their transport to the lysosomes and ensuring intracellular survival. Interestingly, experiments using si-RNA for AKT1 show it has direct involvement in salmonella induced apoptosis. No apoptosis was observed in cells after down regulation of AKT by si-RNA or inhibition using a specific kinase inhibitor H-89 (Kuijl, Savage et al. 2007). This AKT1 inhibitor is currently undergoing trials as an antimicrobial, specifically for Salmonella typhimurium and M. tuberculosis (Kuijl, Savage et al. 2007).

The refinement of the SAL1 QTL in this study identifies both AKT1 and Siva-1 as plausible candidate genes for future study. The role of Siva-1 in apoptosis highlights the essential process of activation-induced cell death (AICD) and the subsequent down-regulation of the immune response as observed in bovine macrophages (Zuemer et al 2007). Siva-1 may also influence the outcome of the innate immune response by its negative regulation of NF-κB (Gudi, Barkinge et al. 2006). Experiments using Siva-1 knockout Jurkat cells showed significantly enhanced TCR-mediated activation of the canonical and non-canonical limbs of the NF-kB pathway. In addition, Gudi et al (2006) found that loss of endogenous Siva-1 resulted in an increased expression of anti-apoptotic genes such as Bcl-xl and FLIP, with consequent implications for peripheral tolerance and innate immunity (Gudi, Barkinge et al. 2006).

The serine/threonine kinase AKT1 is also involved in cellular survival pathways, primarily by inhibiting apoptotic processes. Survival factors can suppress apoptosis in a transcription-independent manner by activating AKT1, which then phosphorylates and inactivates components of the apoptotic machinery. AKT1 can also activate NF-κB via regulating IκB kinase (IKK), thus resulting in transcription of pro-survival genes and having a direct result on the pro-inflammatory response. The hijacking of this pathway by salmonella provides clear evidence for its direct involvement in bacterial proliferation (Madrid, Wang et al. 2000).

To summarise, the inventors confirm that the SAL1 is a significant disease resistance locus for Salmonellosis. Furthermore, with access to genomic sequence and high density SNPs for the chicken genome the inventors have been able to refine the QTL and identify potential candidate genes that may have a significant contribution to salmonella disease resistance. Two functional and positional candidate genes are siva-1 and AKT1.

### Example 2

### Construction of a Vector Comprising Part of the SAL1 Locus

A chicken with a genotype associated with resistance to bacterial infection by Salmonella at a marker of the SAL1 locus which lies between 54.0 MB to 54.8 MB on Chromosome 5 is identified and selected.

A genomic DNA fragment comprising part of the SAL1 locus from this chicken is obtained by restriction digestion of the genomic DNA and identified by Southern blot analysis. The genomic DNA fragment comprising part of the SAL1 locus is isolated from a gel and a vector comprising said DNA fragment is constructed by ligating said DNA fragment into the vector.

The vector may be used to generate transgenic chickens.

### REFERENCES

Ables, G. P., D. Takamatsu, et al. (2001). “The roles of Nramp1 and Tnfa genes in nitric oxide production and their effect on the growth of Salmonella typhimurium in macrophages from Nramp1 congenic and tumor necrosis factor-alpha−/− mice.” J Interferon Cytokine Res 21(1): 53-62.
Bumstead, N. and P. Barrow (1993). “Resistance to Salmonella gallinarum, S. pullorum, and S. enteritidis in inbred lines of chickens.” Avian Dis 37(1): 189-93.
Bumstead, N., L. I. Messer, et al. (1987). “Use of ev loci as a measure of inbreeding in domestic fowls.” Br Poult Sci 28(4): 717-25.
Gudi, R., J. Barkinge, et al. (2006). “Siva-1 negatively regulates NF-kappaB activity: effect on T-cell receptor-mediated activation-induced cell death (AICD).” Oncogene 25(24): 3458-62.
Haley, C. S. and S. A. Knott (1992). “A simple regression method for mapping quantitative trait loci in line crosses using flanking markers.” Heredity 69(4): 315-24.
Hillier (2004). “Sequence and comparative analysis of the chicken genome provide unique perspectives on vertebrate evolution.” Nature 432(7018): 695-716.
Hu, J., N. Bumstead, et al. (1997). “Resistance to salmonellosis in the chicken is linked to NRAMP1 and TNC.” Genome Res 7(7): 693-704.
Kaiser, M. G. and S. J. Lamont (2001). “Genetic line differences in survival and pathogen load in young layer chicks after Salmonella enterica serovar enteritidis exposure.” Poult Sci 80(8): 1105-8.
Kaiser, M. G. and S. J. Lamont (2002). “Microsatellites linked to Salmonella enterica Serovar Enteritidis burden in spleen and cecal content of young F1 broiler-cross chicks.” Poult Sci 81(5): 657-63.
Knodler, L. A., B. B. Finlay, et al. (2005). “The Salmonella effector protein SopB protects epithelial cells from apoptosis by sustained activation of Akt.” J Biol Chem 280(10): 9058-64.
Kuijl, C., N. D. Savage, et al. (2007). “Intracellular bacterial growth is controlled by a kinase network around PKB/AKT1.” Nature 450(7170): 725-30.
Legare, M. E., F. S. Bartlett, 2nd, et al. (2000). “A major effect QTL determined by multiple genes in epileptic EL mice.” Genome Res 10(1): 42-8.
Lembertas, A. V., L. Perusse, et al. (1997). “Identification of an obesity quantitative trait locus on mouse chromosome 2 and evidence of linkage to body fat and insulin on the human homologous region 20q.” J Clin Invest 100(5): 1240-7.
Leveque, G., V. Forgetta, et al. (2003). “Allelic variation in TLR4 is linked to susceptibility to Salmonella enterica serovar Typhimurium infection in chickens.” Infect Immun 71(3): 1116-24.
Madrid, L. V., C. Y. Wang, et al. (2000). “Akt suppresses apoptosis by stimulating the transactivation potential of the RelA/p65 subunit of NF-kappaB.” Mol Cell Biol 20(5): 1626-38.
Malek, M. and S. J. Lamont (2003). “Association of INOS, TRAIL, TGF-beta2, TGF-beta3, and IgL genes with response to Salmonella enteritidis in poultry.” Genet Sel Evol 35 Suppl 1: S99-111.
Mariani, P., P. A. Barrow, et al. (2001). “Localization to chicken chromosome 5 of a novel locus determining salmonellosis resistance.” Immunogenetics 53(9): 786-91.
Monack, D. M., W. W. Navarre, et al. (2001). “Salmonella-induced macrophage death: the role of caspase-1 in death and inflammation.” Microbes Infect 3(14-15): 1201-12.
O'Brien, G. C., J. H. Wang, et al. (2005). “Bacterial lipoprotein induces resistance to Gram-negative sepsis in TLR4-deficient mice via enhanced bacterial clearance.” J Immunol 174(2): 1020-6.
Poltorak, A., I. Smirnova, et al. (1998). “Genetic and physical mapping of the Lps locus: identification of the toll-4 receptor as a candidate gene in the critical region.” Blood Cells Mol Dis 24(3): 340-55.
Seaton, G., C. S. Haley, et al. (2002). “QTL Express: mapping quantitative trait loci in simple and complex pedigrees.” Bioinformatics 18(2): 339-40.
Sebastiani, G., L. Olien, et al. (1998). “Mapping of genetic modulators of natural resistance to infection with Salmonella typhimurium in wild-derived mice.” Genomics 47(2): 180-6.
Soller, M., S. Weigend, et al. (2006). “Strategies to assess structural variation in the chicken genome and its associations with biodiversity and biological performance.” Poult Sci 85(12): 2061-78.
Tilquin, P., P. A. Barrow, et al. (2005). “A genome scan for quantitative trait loci affecting the Salmonella carrier-state in the chicken.” Genet Sel Evol 37(5): 539-61.
Todd, J. A. (1999). “From genome to aetiology in a multifactorial disease, type 1 diabetes.” Bioessays 21(2): 164-74.
Wang, S. S., W. Shi, et al. (2007). “Mapping, genetic isolation, and characterization of genetic loci that determine resistance to atherosclerosis in C3H mice.” Arterioscler Thromb Vasc Biol 27(12): 2671-6.
Wigley, P., S. D. Hulme, et al. (2002). “In vivo and in vitro studies of genetic resistance to systemic salmonellosis in the chicken encoded by the SAL1 locus.” Microbes Infect 4(11): 1111-20.
Wong, G. K., B. Liu, et al. (2004). “A genetic variation map for chicken with 2.8 million single-nucleotide polymorphisms.” Nature 432(7018): 717-22.
Yoon, Y., Z. Ao, et al. (1999). “Murine Siva-1 and Siva-2, alternate splice forms of the mouse Siva gene, both bind to CD27 but differentially transduce apoptosis.” Oncogene 18(50): 7174-9.
Zhou, H. and S. J. Lamont (2003). “Chicken MHC class I and II gene effects on antibody response kinetics in adult chickens.” Immunogenetics 55(3): 133-40.
Zuerner R. L., Heidari M., Elliott M. K., Alt D. P. & Neill J. D. (2007) Papillomatous digital dermatitis spirochetes suppress the bovine macrophage innate immune response. Vet Microbiol 125, 256-64.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

1. A method of identifying an animal having a genotype associated with resistance to bacterial infection comprising the steps of:
(a) providing a sample from said animal;
(b) determining the alleles at one or more markers of the SAL1 locus from the sample to identify the genotype of the marker, wherein said SAL1 locus lies between 54.0 MB to 54.8 MB of chicken Chromosome 5 or an equivalent thereof; and
(c) determining whether the genotype is a genotype associated with resistance to bacterial infection.

2. The method according to claim 1 wherein said claim further comprises the step of:
(d) selecting an animal having the genotype associated with resistance to bacterial infection.

3. A method of identifying a genotype associated with resistance to bacterial infection comprising the steps of:
(a) providing samples from more than one animal;
(b) determining from the samples that there is an allelic variant at a marker of the SAL1 locus to identify a polymorphic marker, wherein said SAL1 locus lies between 54.0 MB to 54.8 MB of chicken Chromosome 5 or an equivalent thereof;
(c) determining that a genotype of the polymorphic marker is associated with resistance to bacterial infection.

4. A method according to claim 1 wherein the genotype associated with resistance to bacterial infection is a genotype associated with resistance to salmonellosis.

5. A method for predicting the response of an animal to infection by bacteria comprising the steps of:
(a) providing a sample from said animal;
(b) determining the alleles at one or more markers of the SAL1 locus from the sample to identify the genotype of the marker, wherein said SAL1 locus lies between 54.0 MB to 54.8 MB of chicken Chromosome 5 or an equivalent thereof; and
(c) determining whether the genotype is a genotype associated with resistance to bacterial infection to predict the response of an animal to infection by bacteria.

6. The method according to claim 5 wherein said bacteria is a Salmonella.

7. The method according to claim 1 wherein the one or more markers are selected from the group consisting of:
the single nucleotide polymorphism SNP2 (rs16511470) on chicken Chromosome 5;
the microsatellite marker ADL166 (UniSTS:71823) on chicken Chromosome 5;
a polymorphism in the nucleotide sequence ENSGALG00000011620 (AKT1) on chicken Chromosome 5; and
a polymorphism in the nucleotide sequence ENSGALG00000011619 (SIVA1) on chicken Chromosome 5.

8. A method for producing an animal which is resistant to bacterial infection or increasing the resistance to bacterial infection of an animal wherein said method comprises the step of replacing at least part of the SAL1 locus with a SAL1 locus or corresponding part thereof from an animal which is resistant to bacterial infection, wherein the SAL1 locus lies between 54.0 MB to 54.8 MB of chicken Chromosome 5 or an equivalent thereof.

9. The method according to claim 8 wherein said bacterial infection is infection by Salmonella.

10. The method according to claim 1 wherein said animal is a fowl.

11. The method according to claim 10 wherein said fowl is a chicken.

12-16. (canceled)

17. A kit for identifying in a sample the genotype of one or more markers at the SAL1 locus, wherein said SAL1 locus lies between 54.0 MB to 54.8 MB of chicken Chromosome 5 or an equivalent thereof, and wherein said kit comprises a means for determining alleles of one or more markers wherein said one or more markers are selected from the group consisting of:
the single nucleotide polymorphism SNP2 (rs16511470) on chicken Chromosome 5;
the microsatellite marker ADL166 (UniSTS:71823) on chicken Chromosome 5;
a polymorphism in the nucleotide sequence ENSGALG00000011620 (AKT1) on chicken Chromosome 5; and
a polymorphism in the nucleotide sequence ENSGALG00000011619 (SIVA1) on chicken Chromosome 5.

18. The kit according to claim 17 wherein said kit further comprises instructions for identifying the genotype of said one or more markers.

19. The kit according to claim 17 wherein said marker is a single nucleotide polymorphism SNP2 (rs16511470) on chicken Chromosome 5, and the means for determining alleles of said marker is one or more oliognucleotide primers or probes selected from the group consisting of 5′-ATCTCAGCCCCATAAAAACGC-3′ (SEQ ID NO 44), 5′-TAGAGTCGGGGTATTTTTGCG-3′ (SEQ ID NO 45), 5′-ATCTCAGCCCCATAAAAACGT-3′ (SEQ ID NO 46) and 5′-TAGAGTCGGGGTATTTTTGCA-3′ (SEQ ID NO 47); and/or wherein said marker is a the microsatellite marker ADL166 (UniSTS:71823) on chicken Chromosome 5, and the means for determining alleles of said marker is oliognucleotide primers or probes selected from the group consisting of 5′-TGCCAGCCCGTAATCATAGG-3′ (SEQ ID NO 40), 5′-AAGCACCACGACCCAATCTA-3′ (SEQ ID NO 41), ACGGTCGGGCATTAGTATCC-3′ (SEQ ID NO 48) and 5′-TTCGTGGTGCTGGGTTAGAT-3′ (SEQ ID NO 49.

20. An array wherein said array comprises one or more oligonucleotide probes capable of determining in a sample the alleles at one or more markers wherein said one or more markers are selected from the group consisting of:
the single nucleotide polymorphism SNP2 (rs16511470) on chicken Chromosome 5;
the microsatellite marker ADL166 (UniSTS:71823) on chicken Chromosome 5;
a polymorphism in the nucleotide sequence ENSGALG00000011620 (AKT1) on chicken Chromosome 5; and
a polymorphism in the nucleotide sequence ENSGALG00000011619 (SIVA1) on chicken Chromosome 5.

21-23. (canceled)

24. An isolated oligonucleotide primer or oligonucleotide probe wherein said oligonucleotide probe or oligonucleotide primer is selected from the group consisting of SEQ ID Nos 39 to 41 and 44 to 49.

25-35. (canceled)

36. A method according to claim 3 wherein the genotype associated with resistance to bacterial infection is a genotype associated with resistance to salmonellosis.

37. The method according to claim 3 wherein the one or more markers are selected from the group consisting of:
the single nucleotide polymorphism SNP2 (rs16511470) on chicken Chromosome 5;
the microsatellite marker ADL166 (UniSTS:71823) on chicken Chromosome 5;
a polymorphism in the nucleotide sequence ENSGALG00000011620 (AKT1) on chicken Chromosome 5; and
a polymorphism in the nucleotide sequence ENSGALG00000011619 (SIVA1) on chicken Chromosome 5.

38. The method according to claim 5 wherein the one or more markers are selected from the group consisting of:
the single nucleotide polymorphism SNP2 (rs16511470) on chicken Chromosome 5;
the microsatellite marker ADL166 (UniSTS:71823) on chicken Chromosome 5;
a polymorphism in the nucleotide sequence ENSGALG00000011620 (AKT1) on chicken Chromosome 5; and
a polymorphism in the nucleotide sequence ENSGALG00000011619 (SIVA1) on chicken Chromosome 5.
